(19) United States
(12) Patent Application Publication
(10) Pub. No.: US 2006/0106002 A1
(43) Pub. Date: May 18, 2006

(54) Therapeutic agent for steroid depending-and/or steroid resistant- ulcerative colitis

(75) Inventors: Toshifumi Ohkusa, Tokyo (JP); Nobuhiro Satoh, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Juntendo Educational Foundation, Tokyo (JP); Ajinomoto Co., Inc., Tokyo (JP)

(21) Appl. No.: 11/225,047

(22) Filed: Sep. 14, 2005

(30) Foreign Application Priority Data
Oct. 20, 2004 (JP) 2004-305964

(51) Int. Cl. A61K 31/4164; A61K 31/43; A61K 31/65

(52) U.S. Cl. 514/152; 514/192; 514/398

(57) ABSTRACT

The present invention is directed to a pharmaceutical composition for the treatment of steroid depending and/or steroid resistive ulcerative colitis. The pharmaceutical composition, which is administered to treat steroid depending and/or steroid resistive ulcerative colitis, contains, as the active ingredients, at least one compound selected from a group consisting of tetracyclines, penicillins, and nitroimidazoles.

### FIELD OF THE INVENTION

The present invention relates to a pharmaceutical composition, a packaged pharmaceutical product, and a method for the treatment of steroid depending and/or steroid resistive ulcerative colitis.

### BACKGROUND ART

Ulcerative colitis is the disease that tends to produce ulcers primarily in mucous membrane to recurrently induce stomach ache, diarrhea, hematochezia, mucoidal stools, and conventionally, medicaments to treat these conditions include antiinflammatories such as steroid hormone, salazosulfapyridine, 5-ASA (5-aminosalicylic acid), and so on. Patients, when medicated with these drugs, clinically recovers from the conditions, but, in most cases, have a relapse over the long time. In many patients, the conditions, even if relieved, are endoscopically and pathologically diagnosed as still being inflammatory. In other words, clinically fully effective medicines of this disease have not been found yet.

Under the circumstances, a newly developed medicament for ulcerative colitis is an antibacterial agent attacking entrobacteria as targets (see “All about Inflammatory Enteropathy for Clinicians: The Up-to-Date Strategies of the Treatment for Ulcerative colitis and Crohn's Disease” edited by Masakazu Takazoe, MedicalVew Inc., (2002)). For example, Satoh and Ohkusa observed that the patients suffered from ulcerative colitis, compared with healthy people and patients of other enteropathies, have more fusobacterium varium stuck to the gut mucosa and infiltrated into mucus and ulceromucosa, have a higher blood antibody detection ratio to fusobacterium varium and a higher amount of the antibodies, and have a greater latency that ulcerative colitis is resulted from butyric acid derived from fusobacterium varium; and they administered antibacterial drug effective against fusobacterium varium to the patients of ulcerative colitis and made its effect certain (see Japanese Patent un-examined Publication No. 2002-363099; United States Patent un-examined Publication No. 2002/0187152; Gut. 52 (1), pp. 79-83 (2003); and Program of the Annual Meeting of the American Gastroenterological Association and Digestive Disease Week, May 17-22, 2003, Orland, Fla., 544).

However, the development of medicaments and treatment methods for inveterate diseases of steroid depending ulcerative colitis and steroid resistive ulcerosa, which has not been satisfactorily advanced, is still strongly desired. In general treatment for diseases, it is a well-known fact that some medicine effective to patients of minor to mild cases is not necessarily useful to a patient of serious symptoms.

### SUMMARY OF THE INVENTION

Accordingly, it is an object of the present invention to provide a pharmaceutical composition for the treatment of steroid depending and/or steroid resistive ulcerative colitis.

It is another object of the present invention to provide a packaged product for the treatment of steroid depending and/or steroid resistive ulcerative colitis.

It is still another object of the present invention to provide a method for the treatment of steroid depending and/or steroid resistive ulcerative colitis.

The inventors of the present invention dedicated themselves to the study in order to overcome the aforementioned disadvantages in the prior art. As a consequence, they found a hard-to-predict fact that some antibacterial agent effective to fusobacterium varium is significantly useful to treat the inveterate disease of steroid depending or steroid resistive ulcerative colitis, and thus, the present invention is made.

(1) The present invention provides a pharmaceutical composition for the treatment of steroid depending and/or steroid resistive ulcerative colitis, and the invention is characterized in that the pharmaceutical composition contains, as the active ingredients, at least one compound selected from a group consisting of tetracyclines, penicillins, and nitroimidazoles.

(2) The present invention also provides a packaged product for the treatment of steroid depending and/or steroid resistive ulcerative colitis, and the product contains at least one pharmaceutical composition selected from a group consisting of a pharmaceutical composition comprising as the active ingredients tetracyclines, a pharmaceutical composition comprising as the active ingredients penicillins, and a pharmaceutical composition comprising as the active ingredients nitroimidazoles.

(3) The present invention further provides a method for the treatment of steroid depending and/or steroid resistive ulcerative colitis, and the invention is characterized by comprising the step of administering at least one compound selected from a group consisting of tetracyclines, penicillins, and nitroimidazoles, to a patient in need thereof.

As will be recognized in the following descriptions of Examples of the present invention, some specific antibacterial agent effective to fusobacterium varium is useful to treat steroid depending and/or steroid resistive ulcerative colitis, and hence, the pharmaceutical composition, the packaged product, and the method according to the present invention are useful to treat symptoms of steroid depending and/or steroid resistive ulcerative colitis.

Moreover, the treatment method according to the present invention is superior in view of safety. In practicing the treatment method of the present invention, the pharmaceutical composition and the packaged product can be conveniently and safely applied, and thus, they are useful in combination.

### DETAILED DESCRIPTION OF THE INVENTION

The term “steroid depending ulcerative colitis” used herein means recurrent ulcerative colitis due to the reduced dosage of steroid in oral or enema administration or the interruption of the steroid administration, and more specifically, the term covers the recurrent ulcerative colitis patients of which cannot reduce the dosage of steroid for one month or more (i.e., a reduction of the oral dosage of steroid down to 75% to 40% of the amount during the remission administration results in inflammatory conditions recurrently occurring within one month), the ulcerative colitis patients of which have taken steroid orally for one year or more, and the recurrent ulcerative colitis patients of which are intolerable to a gradual reduction of the oral dosage of steroid down to the converted dosage of prednisolone of 15 mg/day after the remission administration is introduced. The term “steroid resistive ulcerative colitis” is irremediable ulcerative colitis patients of which show no recovery from the symptoms after oral or enema administration of steroid, and more specifically, the term covers the ulcerative colitis patients of which cannot be recured from hematochezia visually observed after the oral or enema administration of steroid is conducted for one month or more, and the ulcerative colitis patients of which show no remission after the converted dosage of prednisolone of 30 mg/day is administered for two weeks or longer. The term “steroid” used herein is that which is like adrenocortical hormone used for the treatment of ulcerative colitis, and more specifically, one of clinically available prescriptions of prednisolone, prednisolone sodium phosphate, betamethasone, betamethasone sodium phosphate, and dexamethazone.

“Tetracyclines” are a family of antibacterial substances that have tetracycline stereoparents and show antibacterial behavior because of their tendency to inhibit from synthesizing protein; and more particularly, they include oxytetracycline, chlortetracycline, tetracycline, demethylchlortetracycline, rolitetracycline, doxycycline, minocycline, and metacycline, and tetracycline is the most preferable.

“Penicillins” are penicillin family of antibacterial substances that show antibacterial tendency to inhibit bacterium from synthesizing cell wall; and more specifically, they include benzylpenicillin potassium, ampicillin, amoxicillin, and pivmecillinam hydrochloride, and amoxicillin is the most preferable.

“Nitroimidazoles” are a family of antibacterial substances that have nitroimidazole skeletons and are anti-bacterially active against not only trichomonadidae but anaerobic bacterium; and more specifically, they include metronidazole, tinidazole, and ornidazole, and metronidazole is the most preferable.

The “packaged product” is a kit for therapeutic agent that contains one or more pharmaceutical compositions of the present invention packaged together or packaged separately but packed in combination into a single unit.

The pharmaceutical composition (1) according to the present invention preferably contains two or more of the compounds as the active ingredients;

(4) It more preferably contains three of them, namely, tetracyclines, penicillins, and nitroimidazoles as the active ingredients; and

(5) It still more preferably contains tetracycline, amoxicillin, and metronidazole as the active ingredients.

In the pharmaceutical composition (4) and (5) according to the invention, a mass ratio of the active ingredients, namely, tetracyclines, penicillins, and nitroimidazoles is 0.5˜6:0.5˜6:1; more preferably, 1˜4:1˜4:1; and still more preferably, 1500:1500:750 (i.e., 2:2:1); or any alternative ratio if medically equivalent effects can be attained.

(6) The pharmaceutical composition of the present invention can be formed in a packaged product comprising the pharmaceutical compositions along with printed instructions about how to dose the pharmaceutical composition.

Regarding the packaged product (2) according to the present invention,

(7) It preferably contains printed instructions about how to dose each pharmaceutical composition; and

It further preferably contains two or more of the pharmaceutical compositions.

Also, regarding the packaged product (2) according to the present invention,

(8) It preferably comprises a pharmaceutical composition comprising as the active ingredients tetracyclines, a pharmaceutical composition comprising as the active ingredients penicillins, and a pharmaceutical composition comprising as the active ingredients nitroimidazoles, along with printed instructions about how to dose them.

(9) The packaged product more preferably comprises a pharmaceutical composition comprising as the active ingredient tetracycline, a pharmaceutical composition comprising as the active ingredient penicillin, and a pharmaceutical composition comprising as the active ingredient nitroimidazole, along with printed instructions about how to dose them.

In the packaged products of (8) and (9) according to the invention a mass ratio of the active ingredients, namely, tetracyclines, penicillins and nitroimidazoles, is 0.5˜6:0.5˜6:1, more preferably, 14:1˜4:1, and still more preferably, 1500:1500:750 (i.e., 2:2:1), or any alternative ratio that can attain clinically equivalent effects.

Regarding the treatment method (3) of the present invention,

(10) It preferably includes the step of administering tetracycline, amoxicillin, and metronidazole in combination.

When more than one compounds are administered in the treatment method as identified with (3), the compounds may be dosed at the same time, or otherwise, they may be dosed separately at an appropriate time interval.

In accordance with the present invention, all the active ingredients referred to herein as tetracycline(s), penicillin(s), amoxicillin(s), nitroimidazole(s), metronidazole(s), and so on, include the substances as inherently they are, and pharmaceutically acceptable salts, hydrates, and solvates of the same.

The “printed instructions about how to dose the pharmaceutical composition” used herein include a sheet of printed matter contained in the kit as well as the package itself with such instructions printed therein. The printed instructions contain descriptions that the pharmaceutical composition of the present invention can be or must be dosed to patients suffered from steroid depending and/or steroid resistive ulcerative colitis. The printed instructions may also contain usage, dosage, and warnings in taking the drug.

The pharmaceutically acceptable salts for an acidic group such as carboxyl group in the formula include alkaline metal salts such as an ammonium salt, a sodium salt, a potassium salt, and so on; alkali-earth metal salts such as a calcium salt, a magnesium salt, and so on; organic amine salts such as an aluminum salt, a zinc salt, a triethylamine salt, an ethanolamine salt, a morpholine salt, a piperidine salt, a dicyclohexyl amine salt, and so on; and basic amino acid salts such as an arginine salt, a lysine salt, and so on. Other pharmaceutically acceptable salts for a basic group in the formula include inorganic acid salts such as a hydrochloric acid salt, a sulfuric acid salt, a phosphoric acid salt, a nitric acid salt, a hydrobromic acid salt, and so on; organic carboxylic acid salts such as an acetic acid salt, a citric acid salt, a benzoic acid salt, a maleic acid salt, a fumaric acid salt, a tartaric acid salt, a succinic acid salt, a tannic acid salt, a butyric acid salt, a hibenzic acid salt, a pamoic acid salt, an enanthic acid salt, a decanoic acid salt, a teoclic acid salt, a salicylic acid salt, a lactic acid salt, an oxalic acid salt, a mandelic acid salt, and a malic acid salt, and so on; and organic sulfonic acid salts such as a methanesulfonic acid salt, a benzenesulfonic acid salt, a p-toluenesulfonic acid salt, and so on. To produce these salts, the compound(s) comprising medicinal components are mixed with the required acid or base in an appropriate mass ratio in solvent or dispersant, or alternatively, these salts can be obtained through positive ion exchange or negative ion exchange from other structural formulae of the salts.

The pharmaceutical composition of the present invention can take any form like tablet, powder, pill, granule, capsule, suppository, liquid sugar-coated tablet, depot, syrup, suspension; emulsion, troche, sublingual tablet, plaster, intraoral disintegrator, inhalent, enema, ointment, patch, tape, and ophthalmic solutions, and ordinary preparation adjuvant is added to produce the drug according to the common procedures.

Carriers used for the pharmaceutical composition of the present invention may be any of various organic or inorganic carrier substances well-known as preparation material; they include, for instance, excipient, lubricant, binder, disintegrator, water-soluble macromolecule, basic inorganic salt, solvent in the liquid formula, adjuvant enhancing solubility, suspending agent, isotonicity-enhancing agent, buffer, pain reliever, and so on. Also, as required, additives such as ordinary preservative, antioxidant, food color, sweetener, acidulant, gas-releasing agent, aromatic, and so on can be applied.

The pharmaceutical composition according to the present invention does not have to contain any carrier if unnecessary, and it may be comprised simply of active ingredients, or any pharmaceutically acceptable salt, hydrate, or solvate of the ingredients.

The excipient can be any of lactose, cornstarch, white sugar, glucose, sorbitol, crystalline cellulose, and so on. The binding may be, for example, polyvinyl alcohol, polyvinyl ether, ethyl cellulose, methyl cellulose, acacia gum, Tragacanth gummifer, gelatin, shellac, hydroxypropyl cellulose, hydroxypropyl starch, polyvinyl pyrrolidone, or so on. The disintegrator may be any of potatostarch, agar, gelatin powder, crystalline cellulose, calcium carbonate, sodium bicarbonate, calcium citrate, dextran, pectin, and so on. The lubricant may be, for example, magnesium stearate, talc, polyethylene glycol, silica, hydrogenated vegetable oil, or so on. The food color may be anything that is permitted to add to pharmaceutical products. The flavor/aroma correctives include cocoa powder, peppermint camphor, balmy acid, peppermint oil, bomeo camphor, cinnamon powder, and so forth. Tablets and granules of the drug can be, of course, coated with sugar, gelatin, or any appropriate substance as required.

A preparation for injection may contain pH adjusting agent, buffer, stabilizer, and/or preservative as additives to assume a form appropriate to hypodermic injection, intramuscle injection, and intravenous injection according to common procedures.

Other carriers and additives applied to the pharmaceutical composition of the present invention include those which are set forth in The Encyclopedia of Pharmaceutical Additives 2000 edited by Japan Pharmaceutical Excipients Council, Jiji-Nippoh (2000) (The Encyclopedia of Pharmaceutical Additives 2000 edited by Japan Pharmaceutical Excipients Council, Jiji-Nippoh (2000)).

The pharmaceutical composition of the present invention can be manufactured pursuant to the descriptions disclosed, for example, in “Unit Operations and Opportunities of Pharmaceutical Formulae”, The Development of Pharmaceutics Vol. 1, edited by Nakai, Hirokawa Shoten (1989) or “Designs and Assays of Orally Administered Drugs” edited by Hashida, Yakugyo Jiho-Sha (1995); or rather, it can be manufactured by some simple modification by a person skilled in the art as required.

The packaged product of the present invention can be manufactured by using the above-mentioned or known pharmaceutical composition and any method apparent to any person having ordinary skilled in the art.

When the packaged product of the present invention contains more than one pharmaceutical composition, they may be dosed to a patient at a time or separately at an appropriate time interval.

A dosage of the pharmaceutical composition of the present invention is varied depending upon age, way of administration, formula, and duration of the treatment, and typically, an adult is orally dosed with 0.5 mg to 10 g of active ingredients per day, preferably 3.0 g to 4.5 g. In the case of non-oral administration to an adult, the dosage of the active ingredients per day is 1 μg to 3 g, preferably 10 mg to 3 g, and more preferably 0.5 g to 1 g.

As to the administration of the product of the present invention, an oral administration is desirable. This is because of a better medication compliance.

Preferably, the pharmaceutical composition of the present invention contain anti-inflammatory agent, such as steroid, salazosulfapyridine, 5-ASA (5-aminosalicylic acid), and so on, conventionally used to treat ulcerative colitis.

It is also preferable that the packaged product of the present invention contains the anti-inflammatory pharmaceutical agent, such as steroid, salazosulfapyridine, 5-ASA (5-aminosalicylic acid), and so on, conventionally used to treat ulcerative colitis.

It is further preferable that the treatment method of the present invention includes using in combination the anti-inflammatory pharmaceutical agents, such as steroid, salazosulfapyridine, 5-ASA (5-aminosalicylic acid), and so on, conventionally used to treat ulcerative colitis.

As with the anti-inflammatory agents that can be used in combination with the active ingredients like tetracycline, typically an adult is orally dosed with 0.5 mg to 10 g of active ingredients per day; particularly, 0.5 mg to 50 mg of steroid, preferably 3.0 g to 4.5 g of the same; and 10 mg to 10 g of salazosulfapyridine or 5-ASA, preferably 100 mg to 1 g. In the case of non-oral administration to an adult, the dosage of the active ingredients per day is typically 0.1 mg to 10 g; particularly, 0.1 mg to 100 mg of steroid, preferably 1 mg to 10 mg, and 10 mg to 10 g of salazosulfapyridine or 5-ASA, preferably 100 mg to 1 g. It is desirable that steroid is non-orally administered by means of intravenous injection or enema, and that salazosulfapyridine or 5-ASA is non-orally administered by means of intravenous injection.

### PREFERRED EMBODIMENTS

Preferred embodiments of the present invention will now be detailed. The embodiments are given by way of preferable example, and the invention should not be precisely limited to them.

500 mg (titer) of amoxicillin (Sawacillin and Pacetocin in trade names), 500 mg (titer) of tetracycline (Achromycin V in trade name), and 250 mg (titer) of metronidazole (Fragyl in trade name) were orally administered three times a day to a patient or subject of steroid depending or steroid resistive ulcerative colitis in its aggressive progression, who had been dosed with steroid as in Table 1 below. The administration of the drugs was continued for two weeks on the everyday basis. The therapeutic results were evaluated three months and twelve months after the end of the administration of the anti-inflammatory agents.

The evaluation results are shown in Table 2.

In determining the treatment as being effective (remittance of the conditions was recognized), the conditions were evaluated, referring to the Lichtiger Symptom Score (National English Journal of Medicines 330(26), p1841-5 (1994)), and the determination of “Effective” was given, at the end of the administration of the anti-inflammatory agents, to the subject when his or her condition of glutinous blood comprising stools were unobserved, diarrhea was considerably decreased in frequency, stomach ache and pressure pain were perfectly relieved, and a blood test proved a normalized C reactive protein. The Lichtiger symptom Score is provided in Table 3, and the results of the blood test are given in Table 4. The withdrawal from steroid was regarded as being successful when a recrudescence was unobserved for more than three months after stopping the administration of the anti-inflammatory agents and succeedingly starting to reduce the dosage of steroid.

An endoscopic biopsy was conducted before the therapeutic test and three and twelve months after the administration of the anti-inflammatory agents, and the findings from the endoscopic biopsy and pathological diagnoses are determinations based upon the Classification of Matts (Quarter Journal of Medicines 30, p393-407 (1961)).

TABLE 1
Particulars of the Subject Patients

| ITEMS | PARTICULARS | |
|---|---|---|
| Steroid | Steroid Depending: | 15 Cases |
| | Steroid Resistive: | 7 Cases |
| Average Age | 41 | |
| Duration of Morbidity | 7.2 Years in Average | (0 to 30 Years) |
| Patient Rate of | 9/13 | |
| Males to Females | | |
| Diseased Site of | All over the Large Intestine | 11 Cases |
| Enteritis | Left Side of Large Intestine | 9 Cases |
| | Rectum | 2 Cases |
| Degree of Disease | Serious | 5 Cases |
| | Bad | 16 Cases |
| | Minor | 1 Cases |

Table 1 provides the particulars of the subject patients. The subject patients included herein are those who were suffered from steroid depending or steroid resistive ulcerative colitis.

TABLE 2
Treatment Results

| | | RESULTS | |
|---|---|---|---|
| Steroid Depending | 15 Cases | Effective | 12 Cases |
| | | Successful in | 9 Cases |
| | | Withdrawal from | |
| | | Steroid | |
| Steroid Resistive | 7 Cases | Effective | 7 Cases |
| | | Successful in | 7 Cases |
| | | Withdrawal from | |
| | RR | Steroid | |
| Total | 22 Cases | Effectiveness Ratio | |
| | | (Remission Ratio) | |
| Ratio of the Subject | | | 86% (19/22) |
| Patients | | | |
| Successful in | | Success Ratio | 84% (16/19) |
| Withdrawal from | | | |
| Steroid | | | |

Table 2 provides the grading results of the treatment with the antibiotic preparations according to the present invention. The patients, 19 cases out of 22 cases, successfully remitted from the conditions (Effective). Among these “effective” subjects, a ratio of success in withdrawal from steroid reached as high as a value of 84% to prove the great results of the treatment.

TABLE 3
RESULTANT SYMPTOM SCORES
(WITH REFERENCE TO LICHTIGER SYMPTOM SCORE)

| | Before Dosing | | | |
|---|---|---|---|---|
| | with Antibacterial | At the End | 3 Months | 12 Months |
| | Agents | of the Dosing | After | After |
| Mean ± SE | 8.4 ± 0.8 | 3.8 ± 0.4 | 3.3 ± 0.6 | 2.8 ± 0.4 |
| p value | | 0.0001 | 0.0001 | 0.0022 |

Table 3 provides the resulting symptom scores. After the treatment, significant improvement of the conditions was observed compared with the conditions before the treatment of the patients, and the improvement continued till three months and twelve months after that.

TABLE 4
FINDINGS RESULTED FROM BLOOD TEST

| | Before Dosing | | | |
|---|---|---|---|---|
| | with | At the End | | |
| | Antibacterial | of the | 3 Months | 12 Months |
| | Agents | Dosing | After | After |
| C Reactive | 1.5 ± | 0.3 ± | 0.9 ± | 0.7 ± |
| Protein | 0.4 | 0.1** | 0.4 | 0.3** |
| (mg/dL) | | | | |
| Erythrocyte | 23 ± | 15 ± | 18 ± | 20 ± |
| Sedimentation | 4 | 2* | 4* | 4 |
| Rate | | | | |
| (mm/1 h) | | | | |
| Leukocyte | 7742 ± | 6421 ± | 6973 ± | 6642 ± |
| Count | 467 | 378** | 476 | 672** |
| (×10⁶/L) | | | | |
| Red Blood | 395 ± | 400 ± | 432 ± | 417 ± |
| Count | 15 | 13 | 10* | 10 |
| (×10¹⁰/L) | | | | |
| Hemoglobin | 11.4 ± | 11.6 ± | 12.5 ± | 12.3 ± |
| (g/dL) | 0.5 | 0.4 | 0.3** | 0.4 |
| Thrombocyte | 33.7 ± | 33.0 ± | 30.1 ± | 30.6 ± |
| Count | 2.8 | 2.7 | 2.0 | 2.8* |
| (×10¹⁰/L) | | | | |
| Total | 6.4 ± | 6.3 ± | 6.9 ± | 7.0 ± |
| Protein | 0.2 | 0.2 | 0.1** | 0.1 |
| (g/dL) | | | | |
| Albumin | 3.6 ± | 3.6 ± | 4.0 ± | 4.0 ± |
| (g/dL) | 0.1 | 0.1 | 0.1 | 0.1 |

*p < 0.05,
**p < 0.01 Significant difference was observed from the same subject group before the treatment

Table 4 provides the resultant findings from the blood test. Among the items of tested substances, significant improvement was observed for all but the albumin level, namely, values of C reactive protein, erythrocyte sedimentation rate, leukocyte count, red blood count, hemoglobin, thrombocyte count, and total protein, after the treatment, three months after the treatment, and/or twelve months after the treatment.

TABLE 5
FINDINGS RESULTED FROM ENDOSCOPY
(BASED UPON THE CLASSIFICATIONS OF MATTS)

| | Before Dosing | | |
|---|---|---|---|
| | with Antibacterial | | |
| | Agents | 3 Months After | 12 Months After |
| Mean ± SE | 3.7 ± 0.1 | 2.5 ± 0.3 | 2.8 ± 0.4 |
| p value | | 0.0029 | 0.0108 |

Table 5 provides the resultant findings from the endoscopic inspections. Three months after the treatment, it was observed the conditions of the patients were improved, compared with the results of the inspections before the treatment, and the improvement continued till twelve months after the treatment.

What is claimed is:

1. A pharmaceutical composition for the treatment of steroid depending and/or steroid resistive ulcerative colitis, comprising as the active ingredients at least one compound selected from a group consisting of tetracyclines, penicillins, and nitroimidazoles.

2. A packaged product for the treatment of steroid depending and/or steroid resistive ulcerative colitis, comprising at least one pharmaceutical composition selected from a group consisting of a pharmaceutical composition comprising as the active ingredients tetracyclines, a pharmaceutical composition comprising as the active ingredients penicillins, and a pharmaceutical composition comprising as the active ingredients nitroimidazoles.

3. A method for the treatment of steroid depending and/or steroid resistive ulcerative colitis, comprising the step of administering at least one compound selected from a group consisting of tetracyclines, penicillins, and nitroimidazoles, to a patient in need thereof.

4. The pharmaceutical composition as defined in claim 1, further comprising pharmaceutically acceptable carrier or excipient.

5. The pharmaceutical composition as defined in claim 1, wherein the tetracyclines are selected from a group consisting of oxytetracycline, chlortetracycline, tetracycline, demethylchlortetracycline, rolitetracycline, doxycycline, minocycline, and metacycline.

6. The pharmaceutical composition as defined in claim 1, wherein the penicillins are selected from a group consisting of benzylpenicillin potassium, ampicillin, amoxicillin, and pivmecillinam hydrochloride.

7. The pharmaceutical composition as defined in claim 1, wherein the nitroimidazoles are selected from a group consisting of metronidazole, tinidazole, and ornidazole.

8. The pharmaceutical composition as defined in claim 1, comprising as the active ingredients tetracyclines, penicillins, and nitroimidazoles.

9. The pharmaceutical composition as defined in claim 8, comprising tetracyclines, penicillins, and nitroimidazoles in the mass ratio of 0.5˜6:0.5˜6:1.

10. The pharmaceutical composition as defined in claim 8, comprising as the active ingredients tetracycline, amoxicillin, and metronidazole.

11. A packaged product for the treatment of steroid depending and/or steroid resistive ulcerative colitis, comprising the pharmaceutical composition as defined in claim 1, and printed instructions on how to dose the same.

12. The packaged product as defined in claim 2, further comprising printed instructions on how to dose each pharmaceutical composition.

13. The packaged product as defined in claim 12, which comprises a pharmaceutical composition comprising as the active ingredients tetracyclines, a pharmaceutical composition comprising as the active ingredients penicillins, a pharmaceutical composition comprising as the active ingredients nitroimidazoles, and their printed instructions on how to dose the same.

14. The packaged product as defined in claim 13, which comprises a pharmaceutical composition comprising as the active ingredients tetracyclines, a pharmaceutical composition comprising as the active ingredients amoxicillins, a pharmaceutical composition comprising as the active ingredients metronidazoles, and their printed instructions on how to dose the same.

15. The method for the treatment as defined in claim 3, wherein the tetracyclines are selected from a group consisting of oxytetracycline, chlortetracycline, tetracycline, demethylchlortetracycline, rolitetracycline, doxycycline, minocycline, and metacycline.

16. The method for the treatment as defined in claim 3, wherein the penicillins are selected from a group consisting of benzylpenicillin potassium, ampicillin, amoxicillin, and Pivmecillinam Hydrochloride.

17. The method for the treatment as defined in claim 3, wherein the nitroimidazoles are selected from a group consisting of metronidazole, tinidazole, and ornidazole.

18. The method for the treatment as defined in claim 3, wherein the tetracyclines, the penicillins, and the nitroimidazoles are administered in combination.

19. The method for the treatment as defined in claim 18, wherein the tetracyclines, the penicillins, and the nitroimidazoles are administered in the mass ratio of 0.5˜6:0.56:1.

20. The method for the treatment as defined in claim 3, wherein tetracyclines, amoxicillin, and metronidazole are administered in combination.
